(19) United States
(12) Patent Application Publication
(10) Pub. No.: US 2018/0064153 A1
(43) Pub. Date: Mar. 8, 2018

(54) Methods of Protecting Against Brain Injury

(71) Applicant: Natural Alternatives International, Inc., Carlsbad, CA (US)

(72) Inventor: Jay R. Hoffman, Oviedo, FL (US)

(21) Appl. No.: 15/697,104

(22) Filed: Sep. 6, 2017

Related U.S. Application Data
(60) Provisional application No. 62/384,807, filed on Sep. 8, 2016.

(51) Int. Cl. A23L 33/175; A23L 33/00; A61B 5/00

(52) U.S. Cl. CPC A23L 33/175; A61B 5/4064; A23L 33/30

(57) ABSTRACT

The present disclosure provides methods of maintaining brain function, memory function and reducing the prevalence of mild traumatic brain injury in an individual at risk of experiencing a mild traumatic brain injury. The present disclosure also provides methods for administering various human dietary supplements to individuals at risk of experiencing a mild traumatic brain injury.

### INCORPORATION BY REFERENCE TO RELATED APPLICATIONS

This application claims priority to of U.S. Provisional Application No. 62/384,807 filed Sep. 8, 2016, the contents of which is incorporated by reference herein in its entirety.

### BACKGROUND

This application claims priority to of U.S. Provisional Application No. 62/384,807 filed Sep. 8, 2016, the contents of which is incorporated by reference herein in its entirety.

Traumatic brain injury (TBI) occurs when an external force causes temporary or permanent neurologic dysfunction. TBI resulting from explosions have been reported to be the most common injury experienced by combat soldiers injured in the recent conflicts in Iraq and Afghanistan. Warden D. (2006) J. Head Trauma Rehabil. 21:398-402. These injuries can range in spectrum from mild to severe, often from exposure to a high pressure blast. Exposure to a low-pressure blast-wave, however, can also exert significant forces on brain tissue without actual penetration of the skull, and still result in changes in neural function. Mayer A R, et al., (2011) Hum Brain Mapp. 32:1825-1835. These injuries are on the mild end of the TBI spectrum (mTBI) and are the most common form (-75%) of TBI in soldiers returning from combat operations. Warden D. (2006) J. Head Trauma Rehabil. 21:398-402; Marshall K R, et al., (2012) Mil Med. 177:67-75.

Oxidative stress and inflammation in the brain may contribute to the cognitive and neurodegeneration associated with mTBI. Aungst S L, et al., (2014) J Cereb Blood Flow Metab. 34:1223-1232; Perez-Polo J R, et al., (2015) J Neurosci Res. 94:549-561; Yang S H, et al., (2013) J Surg Res. 184:981-988. Elevation in brain carnosine may increase antioxidant capacity potentially preventing neurodegeneration associated with the inflammatory response. In addition, reducing the oxidative response may also contribute to preserving brain derived neurotrophic factor (BDNF) expression.

Neuropeptide Y (NPY) is a neuropeptide that is widely distributed in the central nervous system. Recent investigations have demonstrated that NPY regulation is associated with behavioral resilience to stress in rodent models of PTSD. Cohen H, et al., (2012) Neuropsychopharmacology 37:350-363; Hoffman J R, et al., (2015) Medicine and Science in Sports and Exercise, 47:2043-2052. One of the primary roles of BDNF and NPY is to provide neuroprotection and/or neurotrophic action in various brain segments. Cowansage K K, et al., (2010) Curr Mol Pharmacol. 3:12-29; Croce N, et al., (2012) ACS Chem. Neurosci. 3:312-318. A decrease in cognitive function and memory is a clinical feature associated with mTBI that may result from a dysregulation of neurotrophin expression. Kaplan G B, et al., (2010) Behav Pharmacol. 21:427-437. mTBI involves alterations in dendritic remodeling, and neurogenesis within the hippocampal and prefrontal cortical regions. Although the mechanism is not completely understood, evidence does suggest that elevations in cytokine inflammatory markers may contribute to neuronal disruption in rodent models of mTBI. Perez-Polo J R, et al., (2015) J Neurosci Res. 94:549-561; Yang S H, et al., (2013) J Surg Res. 184:981-988. During a traumatic event, microglia are activated from a resting state and migrate to the site of injury at which they release a variety of inflammatory mediators and participate in phagocytosis. Jacobowitz D M, et al., (2012) Brain Res. 1465:80-89. Glial fibrillary acidic protein (GFAP) is an astrocyte protein that is elevated following brain injury. Perez-Polo J R, et al., (2015) J Neurosci Res. 94:549-561; Yang S H, et al., (2013) J Surg Res. 184:981-988; Kochanek A R, et al., (2006) Dev Neurosci. 28:410-419; Hylin M J, et al., (2013) J Neurotrauma. 30:702-715. Elevations in GFAP are thought to be representative of astrocytes providing trophic support to assist in the recovery processes. Dougherty K D, et al., (2000) Neurobiol Dis. 7:574-585. Blast associated neurodegeneration has also been associated with an increased expression of phosphorylated tau protein. Du X, et al., (2016) Oxidative medicine and cellular longevity, Article ID 4159357; Goldstein L E, et al., (2012) Science translational medicine, 4:134ra60. Mechanisms resulting in increases in tau protein expression are not well-understood, but are thought to be related to increases in inflammation. Bhaskar K, et al., (2010) Neuron, 68:19-31.

A concussion is a traumatic brain injury that alters the way the brain functions. Effects are usually temporary but can include headaches and problems with concentration, memory, balance and coordination. Concussions are common, particularly among athletes that play a contact sport, such as football, rugby, ice hockey and boxing. Most concussive traumatic brain injuries are mild.

Concussion is a head injury with a temporary loss of brain function, and causes a variety of physical, cognitive, and emotional symptoms, which may not be recognized if subtle. A variety of signs accompany concussion including somatic (such as headache), cognitive (such as feeling in a fog), emotional (such as emotional changeability), physical signs (such as loss of consciousness or amnesia), behavioral changes (such as irritability), cognitive impairment (such as slowed reaction times), and/or sleep disturbances. Common causes include sports injuries, bicycle accidents, car accidents, and falls, the latter two being the most frequent causes among adults. In addition to a blow to the head, concussion may be caused by acceleration forces without a direct impact, and on the battlefield, mTBI is a potential consequence of nearby explosions.

Headache is the most common physical mTBI symptom. Others include dizziness, vomiting, nausea, lack of motor coordination, difficulty balancing, or other problems with movement or sensation. Visual symptoms include light sensitivity, seeing bright lights, blurred vision, and double vision. Tinnitus, or a ringing in the ears, is also commonly reported. Cognitive symptoms include confusion, disorientation, and difficulty focusing attention. Loss of consciousness may occur, but is not necessarily correlated with the severity of the concussion if it is brief. Post-traumatic amnesia, in which events following the injury cannot be recalled, is a hallmark of concussion. Confusion, another concussion hallmark, may be present immediately or may develop over several minutes. A person may repeat the same questions, be slow to respond to questions or directions, have a vacant stare, or have slurred or incoherent speech.

Other mTBI symptoms include changes in sleeping patterns and difficulty with reasoning, concentrating, and performing everyday activities.

Concussion may be caused by impact forces, in which the head strikes or is struck by something, or impulsive forces, in which the head moves without itself being subject to blunt trauma, for example, a violent shaking of the head an body when the chest hits something and the head snaps forward.

### SUMMARY OF THE INVENTION

In some embodiments, the present disclosure provides a method of reducing the prevalence of mild traumatic brain injury (“mTBI”) in an individual comprising administering a dietary supplement containing a free amino acid beta-alanine to an individual at risk of experiencing mTBI, thereby reducing the prevalence of mTBI in said individual.

In some embodiments, the present disclosure provides a method of maintaining brain function in an individual at risk of mTBI comprising administering a dietary supplement containing a free amino acid beta-alanine to an individual at risk of experiencing mTBI, thereby maintaining brain function in said individual.

In some embodiments, the present disclosure provides a method of maintaining BDNF expression in the hippocampus of an individual at risk of mTBI comprising administering a dietary supplement containing a free amino acid beta-alanine to an individual at risk of experiencing mTBI, thereby maintaining BDNF expression in the hippocampus of said individual.

In some embodiments, the present disclosure provides a method of maintaining GFAP expression in the hippocampus of an individual at risk of mTBI comprising administering a dietary supplement containing a free amino acid beta-alanine to an individual at risk of experiencing mTBI,thereby maintaining GFAP expression in the hippocampus of said individual.

In some embodiments the mTBI is caused by a blast wave. In some embodiments the mTBI is caused by a blow or impact to the head of the individual or by a violent shaking of the head and/or body of the individual. In some embodiments the individual is selected from the group consisting of soldiers, first responders, construction workers, athletes and the like. In some embodiments the athlete is selected from the group consisting of boxers, MMA fighters, wrestlers, judo players, ice hockey players, soccer players, rugby players, football players, gaelic football players, hurlers, lacrosse players, Austrailian Rules football players, motorcycle riders, Formula One race car drivers, Nascar drivers, jockeys and cyclists. In some embodiments, the individual is employed or engaged in high impact or forcefull contact activities. In some embodiments the dietary supplement further comprises a carbohydrate, a creatine, a nitrate, a glycine, or a free amino acid histidine. In some embodiments the free amino acid beta-alanine is in a sustained release formulation.

### BRIEF DESCRIPTION OF THE DRAWINGS

FIG. 1 Schematic depicting study design. The events are presented on the timeline wherein EPM=Elevated plus maze; ASR=Acoustic startle response; MWM=Morris water maze.

FIG. 2 shows the algorithm used to determine the cut-off behavioural criteria. Animals were classified into groups according to degree of response to the stressor. EPM=Elevated Plus Maze; ASR=Acute Startle Response; EBR=Extreme Behavioral Response; MBR=minimal behavioral response (MBR); PBR=partial behavioral response.

FIG. 3 shows the cognitive criteria algorithm. Animals were classified into groups according to degree of cognitive performance in the Morris water maze.

FIG. 4 shows the prevalence rates of animals exhibiting mTBI-like behavior. BA=β-alanine; PL=Placebo; *=Significant difference between the groups.

FIG. 5A shows BDNF Expression at Day 16 Post-Blast Exposure in the CA1 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 5B shows BDNF Expression at Day 16 Post-Blast Exposure in the CA3 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 5C shows BDNF Expression at Day 16 Post-Blast Exposure in the DG section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 6A shows NPY Expression at Day 16 Post-Blast Exposure in the CA1 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 6B shows NPY Expression at Day 16 Post-Blast Exposure in the CA3 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 6C shows NPY Expression at Day 16 Post-Blast Exposure in the DG section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 7A shows GFAP Expression at Day 16 Post-Blast Exposure in the CA1 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 7B shows GFAP Expression at Day 16 Post-Blast Exposure in the CA3 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 7C shows GFAP Expression at Day 16 Post-Blast Exposure in the DG section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 8A shows Tau Expression at Day 16 Post-Blast Exposure in the CA1 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 8B shows Tau Expression at Day 16 Post-Blast Exposure in the CA3 section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 8C shows Tau Expression at Day 16 Post-Blast Exposure in the DG section of the hippocampus. *=significantly different than PL and BA; ̂=significantly different than PL. CTL=control group consisting of animals that were fed a normal diet and not exposed to the blast; PL=animals that were fed a normal diet and were exposed to the blast; BA=animals that were supplemented with β-alanine and exposed to the blast. All data reported as mean±SD.

FIG. 9 shows changes in carnosine and histidine (mM) content in the hippocampus.

### DETAILED DESCRIPTION

The terms mild brain injury, mild traumatic brain injury (mTBI), mild head injury (MHI), minor head trauma, and concussion may be used interchangeably herein, except where a term is specifically discussed.

The methods described herein may be used for many different industries, including, for example, healthcare, military, paramilitary organizations, first responders, such as policemen, firemen and ambulatory personnel, emergency and surgical personnel, sports teams, athletes and many others. The following provides further description of certain embodiments of the invention. As described and claimed herein, certain terms are defined and used interchangeably.

As used herein, “β-alanine”, “beta-alanine”, and “BA” are meant to represent the amino acid beta-alanine that is a free amino acid, or a salt or ester of the free amino acid. As will be understood, these terms are to be used interchangeably except as otherwise specified herein. Unless specified otherwise herein, the use of these interchangeable terms does not encompass beta-alanine as a component of a dipeptide, oligopeptide, or polypeptide. Consequently, a human dietary supplement containing a dipeptide, oligopeptide, or polypeptide without any free amino acid beta-alanine, or an ester or salt thereof, would not be within the scope of the present invention. For example, a dietary supplement of the dipeptide carnosine, or the like, without any free amino acid beta-alanine, would not be within the scope of the present invention. If, however, a human dietary supplement comprises a dipeptide, oligopeptide, or polypeptide in combination with the free amino acid beta-alanine, or an ester or salt thereof, then such human dietary supplement would be within the scope of the present invention, provided the free amino acid beta-alanine, or an ester or salt thereof, is present in an effective amount as defined herein. Naturally, the ester and amide forms of the free amino acid beta-alanine, and their salts, could be used in a similar manner. Additionally, the use of these interchangeable terms in describing the human dietary supplement of the invention does not encompass beta-alanine from a natural or conventional food or food product unless otherwise specifically stated or claimed. Natural or conventional foods or food products include, but are not limited to, beef, pork, chicken, meat extract supplements, and predigested meat/protein supplements, and the various essences of meats. Under these definitions, the term “human dietary supplement” does not encompass, and does not mean, a natural or conventional food or food product, such as chicken meat, meat essences, chicken broth or meat flavoring.

Furthermore, human dietary supplements of the present invention do not encompass pharmaceutical compositions, and the methods of the present invention do not encompass therapeutic treatments. The human dietary supplements described herein are non-pharmaceutical compositions and are used in methods for maintaining brain function, memory function, levels of certain factors in the brain and in reducing the prevalence of mild trumatic brain injury in individuals at risk of mTBI. While the embodiments described herein may utilize pharmaceutical grade ingredients for human consumption and other uses, the human dietary supplements and associated approaches of their use are non-pharmaceutical. The embodiments described herein are intended for use as dietary supplements only.

As used herein, the term “human dietary supplement” is intended to mean a dietary supplement as defined under the Dietary Supplement Health and Education Act of 1994 (“DSHEA”). A human dietary supplement as used herein, also means a dietary supplement that is administered or taken by an individual more than once with the purpose of supplementing the diet to increase and/or maintain a component (e.g., beta-alanine) of the supplement, or a substance comprising a component of the supplement (e.g., carnosine) in the body at a higher level(s) than that naturally occurring through natural or conventional meals. Additionally, human dietary supplement further means an addition to the human diet in a pill, capsule, tablet, powder, or liquid form, which is not part of a natural or conventional food or food product.

As used herein, the term “period of time”, “over time” or “duration of time” means more than a single dosing, taking or administration of the human dietary supplement. More specifically, these terms mean the human dietary supplement is taken one or more times per day over a period of seven or more days, wherein generally no two consecutive days pass without the dietary supplementation and the individual supplements the diet at least 3 or 4 days in any 7 day period, more preferably 4 or more days in any 7 day period, more preferably 5 or more days in any 7 day period, more preferably 6 or more days in any 7 day period, more preferably 7 consecutive days in any 7 day period. For example, the individual can take the dietary supplement every day, wherein the dietary supplement is provided over the course of the day or the individual may take a single dose of the dietary supplement for each day. The individual may also account for non-supplementation days as described above regarding days without supplementation. The period of time described herein can be continued for at least 7 days to about 240 days; preferably about 14 days to about 210 days; more preferably about 21 days to about 180 days; more preferably about 28 days to about 180 days; more preferably about 28 days to about 60 days; even more preferably about 30 days. It will be understood by those of skill in the art, that the period of time can be adjusted by the individual depending on the result to be achieved and/or maintained.

As used herein, the terms “effective amount”,“amount effective to” and similar terms refer to an amount of the supplement required to achieve the results sought and is an amount that is more than contained in the average diet. For example, omnivores consume about 50-300 mg of carnosine per day and the cooking procedures used would lead to a beta-alanine amount lower than this. It will be understood by those skilled in the art that a one time, single dosing of beta-alanine is incapable of achieving an effective amount for the purposes of dietary supplementation with beta-alanine. Furthermore, it will be understood by those skilled in the art that administering a single dose followed with multiple consecutive days (e.g., 3 or more days) of non-dosing or non-supplementation will not achieve the effective amount as described in the invention.

As used herein, the terms “post-traumatic stress disorder”, “PTSD”, etc. refer to, but not limited to, a mental health condition that may be triggered by a traumatic event. The traumatic event may be experienced and/or witnessed. Symptoms may include, but are not limited to, recurrent, unwanted distressing memories of a traumatic event, reliving a traumatic event as if it were happening again (flashbacks), upsetting dreams about a traumatic event, severe emotional distress or physical reactions to something that brings to mind a traumatic event, trying to avoid thinking or talking about a traumatic event, avoiding places, activities or people that bring to mind a traumatic event, negative feelings about oneself or other people, inability to experience positive emotions, feeling emotionally numb, lack of interest in activities once enjoyed, hopelessness about the future, memory problems, such as not remembering important aspects of the traumatic event, difficulty maintaining close relationships, irritability, angry outbursts or aggressive behavior, always being on guard for danger, overwhelming guilt or shame, self-destructive behavior, such as drinking too much or driving too fast, trouble concentrating, trouble sleeping, being easily startled or frightened, and combinations thereof.

Forms and Formulations

Administration of the beta-alanine can be as the free amino acid beta-alanine, wherein the free amino acid beta-alanine is not part of a dipeptide, oligopeptide or polypeptide. The free amino acid beta-alanine can be an ester or salt of beta-alanine. The free amino acid beta-alanine can be in a pill, tablet, capsule, granule or powder form. The free amino acid beta-alanine can be administered as part of a solid, liquid or semi-liquid. The free amino acid beta-alanine can be administered as part of a drink (e.g., sports drink) or a food (e.g., health bar).

The free amino acid beta-alanine may also be administered in a sustained release formulation, wherein the free amino acid beta-alanine is not part of a dipeptide, oligopeptide, or polypeptide. The free amino acid beta-alanine administered in a sustained release formulation may also be present as an ester or salt of the beta-alanine. The sustained release formulation can be in a tablet, capsule, granule or powder form. The sustained release formulation can be administered as part of a solid, liquid or semi-liquid. The sustained release formulation of the free amino acid beta-alanine can be administered as part of a drink (e.g., sports drink) or a food or food matrix (e.g., health or energy bar or energy gel). It has been reported that some individuals may experience a flushing/tingling (i.e., paraesthesia) of the skin when taking β-alanine as a free amino acid. While this sensation may be uncomfortable and/or unwanted, it typically lasts less than 60 minutes. The use of sustained-release forms of beta-alanine has been shown to inhibit, decrease and/or eliminate the flushing/tingling of the skin.

In various embodiments of the present invention, the human dietary supplement may be administered (e.g., consumed or ingested) in combination with other ingredients, except for those other ingredients that are explicitly disclaimed herein. For example, the free amino acid beta-alanine, or an ester or salt thereof, may be administered in combination with creatine, wherein the creatine is in the form of creatine-monohydrate or other acceptable forms of creatine.

In another embodiment, the dietary supplement comprising the free beta-alanine can further comprise one or more carbohydrates, including simple carbohydrates, for example. Additionally, carbohydrates can include starch and/or sugars, e.g., glucose, fructose, galactose, sucrose, and maltose. The sugars or other carbohydrates can be from various forms of honey, molasses, syrup (e.g., corn syrup, glucose syrup), treacle or gels. It will be understood that the human dietary supplement of the invention may comprise one or more carbohydrates in combination with the other ingredients disclosed herein and as part of the forms and formulations defined by the present invention.

In addition, the human dietary supplements of the present invention may further comprise insulin mimics, and/or insulin-action modifiers. Insulin mimics include, but are not limited to, D-pinitol (3-O-methyl-chiroinositol), 4-hydroxy isoleucine, L783,281 (a demethyl-asterriquinone B-1 compound), alpha lipoic acid, R-alpha lipoic acid, guanidiniopropionic acid, vanadium compounds such as vanadyl sulfate or vanadium complexes such as peroxovanadium, and synthetic phosphoinositolglycans (PIG peptides). Insulin-action modifiers that enhance or inhibit the action of insulin in the body, can include, but are not limited to, sulphonylureas, thiazolidinediones, and biguanides. Additionally, the human dietary supplements may comprise insulin stimulating agents (e.g., glucose).

In another embodiment, the dietary supplement comprising the free beta-alanine can further comprise one or more electrolytes and/or vitamins (e.g., vitamins B6, B12, E, C, and thiamin, riboflavin, niacin, folic acid, biotin and pantothenic acid). In other embodiments, the human dietary supplement may comprise lipids, other amino acids, fiber, trace elements colorings, flavors, natural and/or artificial sweeteners, natural health improving substances, anti-oxidants, stabilizers, preservatives, and buffers.

In certain other embodiments, the human dietary supplement of the present invention may comprise other ingredients, for example, anti-oxidants, alpha-lipoic acid, tocotrienols, N-acetylcysteine, co-enzyme Q-10, extracts of rosemary such as carnosol, botanical anti-oxidants such as green tea polyphenols, grape seed extract, COX-1 type inhibitors such as resveratrol, ginkgo biloba, pterostilbene and garlic extracts. Other amino acids such as L-histidine, L-cysteine, L-citrulline, glycine and branched chain amino acids may be added. In some embodiments, a glycine containing molecule may be added, for example, Glycine Propionyl-L-Carnitine. In some embodiments, the present invention may comprise combination with an acetylcholine precursor such as choline chloride or phosphatidylcholine may be desirable, for example, to enhance vasodilation. The invention also provides for human dietary supplements comprising the free amino acid beta-alanine in combination with such other ingredients as minerals and trace elements in any type or form suitable for human consumption. It is convenient to provide calcium and potassium in the form of their gluconates, phosphates or hydrogen phosphates, and magnesium as the oxide or carbonate, chromium as chromium picolinate, selenium as sodium selenite or selenate, and zinc as zinc gluconate.

In certain other embodiments, the human dietary supplement of the present invention may comprise other ingredients, for example, nitrates, such as nitrates of amino acids. The nitrates may also be extracts of naturally occuring nitrates, such as extracts of nitrate containing plants, for example, spinach or beetroot extract.

The ingredients, compounds and components disclosed herein as optionally being in the human dietary supplement comprising the free amino acid beta-alanine, may be in any combination as part of the human dietary supplement. This will be readily understood by those of skill in the field of dietary supplementation and exercise physiology.

Once the levels of beta-alanylhistidine have been increased by use of effective amounts of the human dietary supplement, otherwise known as a loading phase, the dosing can be adjusted to maintain the levels of beta-alanylhistidine necessary to maitain the physiological responses for the purposes of this invention.

In one aspect, the dietary supplement is formulated for one or more servings that can be ingested one or more times per day to achieve an effective amount as required by the present invention. Thus, the total daily intake amount required to meet an effective amount of free beta-alanine, or an ester or salt thereof, can be obtained through a single serving or through multiple smaller servings throughout the day that in total meet the required amount of free beta-alanine, or an ester or salt thereof, to be an effective amount in a total daily intake of the dietary supplement. Therefore, a dietary supplement can be formulated with lower amounts of free beta-alanine, or an ester or salt thereof, for the purpose of multiple servings in a day, wherein the total amount through multiple servings meets the desired total daily intake to be an effective amount as defined by the present invention.

The free amino acid beta-alanine may be administered at a concentration of approximately 100 g/kg of composition. In certain embodiments, the concentration may be approximately 70 g/kg—approximately 130 g/kg, approximately 80 g/kg—approximately 120 g/kg, and/or approximately 90 g/kg—approximately 110 g/kg.

The free amino acid beta-alanine may be provided as a mixture (composition) of free amino acid beta-alanine, or an ester or salt thereof, and one or more other compounds. The one or more other compounds may be, but is not limited to, glucomannan. In certain embodiments, the mixture may be approximately 80% free beta-alanine, or an ester or salt thereof, and approximately 20% glucomannan. In certain embodiments, the percentage of free beta-alanine, or an ester or salt thereof, may be from approximately 50% to approximately 95%, approximately 60% to approximately 90%, and/or approximately 70% to approximately 85%.

The mixture may be provided as a solution. The solution may include approximately 1.0-1.2 grams of composition per approximately 100 ml water.

The total daily intake amount of the free amino acid beta-alanine, or an ester or salt thereof, is in a range of about 0.3 grams (g) to about 16.0 g; preferably about 1.0 g to about 10.0 g; more preferably about 2.0 g to about 8.0 g; and even more preferably about 3.0 g to about 7.0 g. The total daily intake amount of the free amino acid beta-alanine can be in a range of about 3.2 g to about 6.4 g. As described in the present invention, the total daily intake amount in these ranges can be achieved through a single serving formulation comprising the desired effective amount of free beta-alanine. Alternatively, the total daily intake amount in these ranges can be achieved through a formulation for multiple servings, each comprising an amount of the free beta-alanine that when totaled for the day will be within the desired range for a total daily intake delivering an effective amount as defined by the present invention.

Where the dietary supplement is formulated for multiple servings per day within the ranges described herein, it will be understood that there can be 2-12 servings or more, depending on the amounts of free beta-alanine, or ester or salt thereof, in the formulated units. For example, a sustained release tablet comprising 2.0 g of free beta-alanine can be served 3 times per day for a total daily intake of 6.0 g of free beta-alanine. As another example, a formulation comprising 0.5 g of free beta-alanine can be taken 12 times throughout the day for a total daily intake of 6.0 g. This aspect of the present invention applies whether 12 tablets comprising 0.5 g of free beta-alanine are taken at 12 different times throughout the day or if 4 tablets are taken at 3 different times throughout the day. As will be understood in the present invention, it is the total daily intake of the free beta-alanine that must be an effective amount as defined by the present invention. Moreover, the effective amounts in the ranges provided herein account for non-supplementation days as defined by the present invention. Therefore, as long as the individual supplements his/her diet as described herein, the total daily intake of the dietary supplement accounts for non-supplementation days and achieves an effective amount as required over time.

These ranges for total daily intake of free beta-alanine can also account for the various body sizes. Therefore, it will be understood that individuals with smaller body types can take less or more depending on the results to be achieved. Likewise, the ranges for total daily intake account for individuals with larger body types that might require a higher total daily intake to achieve the results. Regardless of body type, the total daily intake of the effective amounts of free beta-alanine, or an ester or salt thereof, in the dietary supplements of the present invention can account for adjustments in amounts based on the individual's body type requirements.

It will also be understood that an effective amount being consumed, as defined herein, can be adjusted up or down as long as the total daily intake of free beta-alanine, or an ester or salt thereof, is maintained within the ranges provided herein and meet the definition of effective amounts of the present invention. For example, an individual taking a dietary supplement of the present invention in a formulation delivering a total daily intake of 6.0 g or 6.4 g, can adjust the level of supplementation down to 1.6 g, 2.0 g, 3.0 g or 3.2 g of a total daily intake of the free amino acid beta-alanine, or an ester or salt thereof. This is referred to as a maintenance phase. It will be understood by those of skill in the art that individuals may achieve a particular result through the dietary supplementation of the present invention and then the individual can opt to reduce the effective amount to a lower effective amount of the present invention to maintain the result. In a converse example, an individual taking a total daily intake of 3.0 g or 3.2 g of the free beta-alanine, or an ester or salt thereof, as an effective amount, can increase the total daily intake of the free beta-alanine to any effective amount within the ranges described herein. For example, the individual could increase the total daily intake from 1.6 g, 2.0 g, 3.0 g or 3.2 g to a total daily intake of 6.0 g or 6.4 g. These examples of adjusting the total daily intake of the free beta-alanine described herein are intended as examples of how an individual can modify the daily intake of beta-alanine, and these examples are not intended to be limiting on the present invention.

It will also be understood from the present disclosure that an individual can cycle the intake of an effective amount of the free beta-alanine between higher and lower total daily intakes of an effective amount of beta-alanine. For example, an individual could take a total daily amount of 6.4 g of the free beta-alanine, or an ester or salt thereof, as an effective amount for a period of 28 days, including non-supplementation days, followed by 28 days of taking 1.6 g of the free beta-alanine, or an ester or salt thereof, as an effective amount, including non-supplementation days, followed by 28 days of taking 6.4 g of the free beta-alanine, or an ester or salt thereof, as an effective amount, including non-supplementation days. It will also be understood that the time periods and total daily intake amounts given in the example of cycling can be adjusted based on the individual's body type requirements.

As will be understood by one of skill in the art through the disclosure of the present invention, other ingredients, e.g., creatine, other amino acids, and carbohydrates, can be present in the human dietary supplement in similar amounts as that described for the free amino acid beta-alanine, or esters or salts thereof.

### EXAMPLES

### General Methods

### Animals

Adult male Sprague-Dawley rats weighing 200-250 gm were habituated to housing conditions for at least seven days. All animals were housed four per cage in a vivarium with stable temperature and a reversed 12-h light/dark cycle, with unlimited access to food and water. In animals randomized to the supplement group (BA), β-alanine was provided with glucomannan in a powder form (80:20 blend). Rats were provided with 100 mg of the powder per kg of body mass (a total of 30 mg of powder was dissolved in 25 ml of water). Placebo (PL) treated rats were provided with the vehicle (glucomannan) at the same relative dose. Animals were handled once daily. All testing was performed during the dark phase in dim red light conditions.

### Experimental Design

Rats were randomly assigned to one of three treatment groups:

(1) Vehicle-treated group+Blast (PL; n=50): rats were fed regular food and water for 30 days and exposed to the low-pressure blast wave; (2) β-alanine +Blast (BA; n=49): rats were fed regular food, provided β-alanine in their water for 30 days and exposed to the low-pressure blast wave; and, (3) Vehicle-treated +unexposed (CTL; n=10); rats were fed regular food for 30 days and were not exposed to the low-pressure blast wave. These animals served as controls for immunofluorescence analysis.

Following the 7-day acclimation period in which all rats received a normal powder diet, they were randomized into the three groups. Following 30-days of either a normal diet or a β-alanine supplemented diet the rats in BA and PL were exposed to a low-pressure blast wave. Diets were maintained until the end of the study.

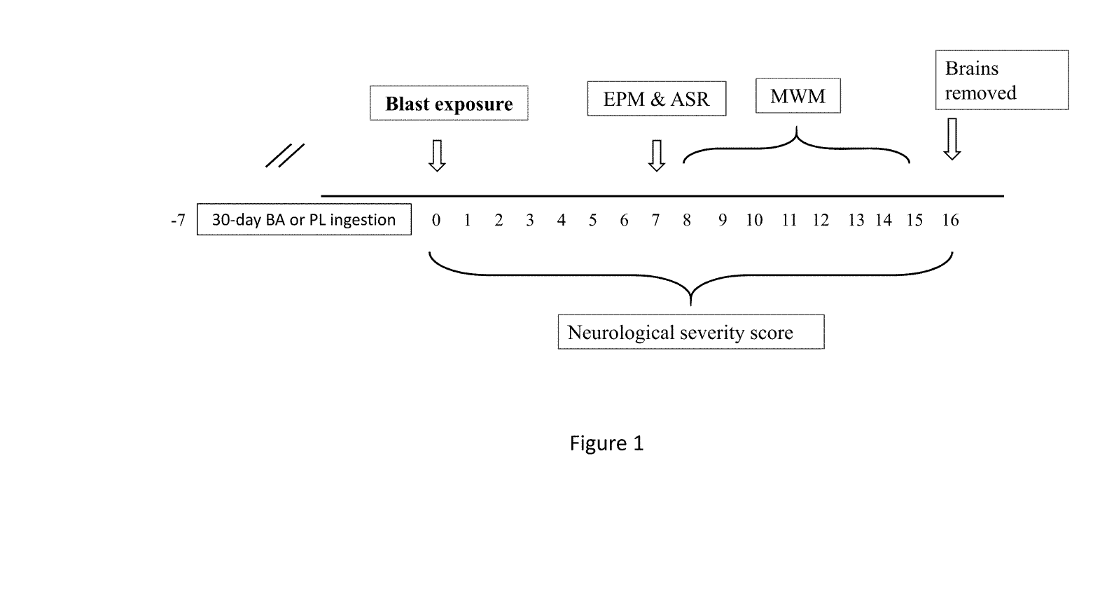

Neurological assessment using the neurological severity score (NSS) was performed one hour following the blast and daily thereafter. Behavior measures were conducted on day seven following the blast. Animals were initially assessed in the elevated plus maze (EPM) followed by the acute startle response (ASR) paradigm one hour later. Spatial memory performance using the Morris water-maze (MWM) test was assessed at eight days post-exposure, for eight consecutive days. Rats were sacrificed 24 hours following the completion of the MWM test. The prevalence rate of rats exhibiting PTSD-like- or mTBI-like responses were calculated from these data. The experimental design is depicted in FIG. 1.

### Blast Wave Exposure

An exploding wire technique was used to generate small-scale cylindrical and spherical blast waves, which simulate the effects of an air blast exposure, such as would be expected from explosive devices in hostile environments. To initiate a low-pressure blast wave explosion a current created by a high voltage power supply (4.2 kV) was generated from a capacitor that was delivered to a thin (0.8 mm diameter, 70 mm in length) knotted copper wire. The discharge current was about 500 kA. When the short, high-current pulse passed through the thin conducting wire, it was rapidly heated, expanded and then evaporated. The rapid expansion generated a strong blast wave, whose strength was controlled by the charging voltage. This method of explosion produced a cylindrical blast wave that simulated a blast wave profile similar to that seen from an explosive device common to the battlefield. In an actual explosion the blast wave causes an acute, short-duration elevation in pressure followed by a negative phase. The exploding wire system has been shown to be capable of simulating this overpressure and negative pressure blast wave profile.

Each rat was restrained in a custom flexible harness located on a tray, which was then placed in the blast wave generation system at a distance of 265 mm from the wire. Movement was restricted to 3-5 cm during the blast exposure. Pressure values were recorded using a Kistler 211B3 piezoelectric pressure transducer mounted on a perpendicular wall. Non-anesthetized rats were subjected to a single blast wave with the head facing the blast without any body shielding, resulting in a full body exposure to the blast wave. Previous research has reported that this low-pressure blast wave results in a mean peak over-pressure of 95 kPa (13.77 psi) (rise time of 0.01 ms) that is sustained for a duration of 0.19 ms and leads to a peak impulse of 10.8×10⁻³ kPa.s⁻¹. The over-pressure wave is followed with a negative pressure wave that is sustained for more than 0.66 ms with a peak negative pressure of −40 kPa (−5.8 psi). This blast protocol is reported to result in a sound pressure level of 193 dB and a light intensity of approximately 5 Mlux, which is similar to that experienced during exposure to a M84 stun grenade at a distance of 1.5 m (3.1 Mlux). The peak overpressure deviations between the different experimental trials was between 1-3%. Following the blast rats were returned to their home cage. Exposure to this experimental blast wave has recently been validated to elicit distinct behavioral and morphological responses modelling mTBI-like, PTSD-like and comorbid mTBI-PTSD-like behaviors. Zuckerman A, et al., (2016), J Neurotrauma. 2016 Feb 17.

### Neurological Severity Score (NSS)

To insure that any damage to the central nervous system (CNS) caused by the blast wave did not result in vast neurological deficits, we employed the NSS. The NSS was performed 1-h following the initial blast wave exposure. NSS assesses somatomotor and somatosensory function by evaluating the animals' activities in motor, sensory, reflexes, beam walking, and beam balancing tasks. Specifically, the following were assessed: ability to exit from a circle (3-point scale), gait on a wide surface (3-point scale), gait on a narrow surface (4-point scale), effort to remain on a narrow surface (2-point scale), reflexes (5-point scale), seeking behavior (2-point scale), beam walking (3-point scale), and beam balance (3- point scale). An observer, who was blind to the different treatment groups, tested the animals. No significant differences between BA and PL were noted in reflex responses, motor coordination, motor strength, or sensory function.

### Behavioral Assessments

All rats underwent a number of different behavioral assessments. All behavioral tests were performed in a closed, quiet, light-controlled room between 10:00-16:00 hr. An results were recorded and analyzed using an EthoVision automated tracking system (Noldus Information Technology, The Netherlands). The behavioral tests included the elevated plus-maze (EPM) and acoustic startle response for anxiety-like/PTSD-like responses and occurred 7-days following initial exposure to the blast wave.

The EPM is a plus-shaped platform with two opposing open and two opposing closed arms (open only towards the central platform and surrounded by 14-cm high opaque walls on three sides). Rats were placed on the central platform facing an open arm and allowed to explore the maze for 5 min. Each session was videotaped and subsequently scored by an independent observer. Arm entry was defined as entering an arm with all four paws. Behaviors assessed were: time spent (duration) in open and closed arms and on the central platform; number of open and closed arm entries; and total exploration (entries into all arms). Total exploration was calculated as the number of entries into any arm of the maze to distinguish between impaired exploratory behavior, exploration limited to closed arms (avoidance) and free exploration. “Anxiety Index”, an index that integrates the ERM behavioral measures, was calculated as follows:

$$Anxiety Index = 1 - \frac{\begin{matrix}\frac{time spent in the open arms}{total time on the maze} + \\ \frac{number of entries to the open arms}{total exploration on the maze}\end{matrix}}{2}$$

Anxiety Index values range from 0-1 where an increase in the index expresses increased anxiety-like behavior.

Acute Startle Response (ASR) was measured using two ventilated startle chambers (SR-LAB system, San Diego Instruments, San Diego, Calif.). The SR-LAB calibration unit was used routinely to ensure consistent stabilimeter sensitivity between test chambers and over time. Each Plexiglas cylinder rested on a platform inside a sound-proofed, ventilated chamber. Movement inside the tube was detected by a piezoelectric accelerometer below the frame. Sound levels within each test chamber were measured routinely using a sound level meter to ensure consistent presentation. Each test session started with a 5-min acclimatization period to background white noise of 68 dB, followed by 30 acoustic startle trial stimuli in six blocks (110 dB white noise of 40 ms duration with 30 or 45 sec inter-trial interval). Behavioral assessment consisted of mean startle amplitude (averaged over all 30 trials) and percent of startle habituation to repeated presentation of the acoustic pulse. Percent habituation defined as the percent change between the response to the first block of sound stimuli and the last block of sound stimuli was calculated as follows:

Percent Habituation=100×((average startle amplitude in block 1)−(average startle amplictude in block 6))/(average startle amplitude in Block 1).

Spatial learning and memory was assessed by performance in a hippocampal-dependent visuospatial learning task in the Morris Water Maze (MWM). Animals were trained in a pool 1.8 m in diameter and 0.6 m high, filled half-way with water at 24°±1° C. A 10-cm square transparent platform was hidden in a constant position in the pool submerged 1-cm below water level. Within the testing room only distal visual-spatial cues were available to the rats for location of the submerged platform. Rats were given four trials per day, to find the hidden platform over four consecutive days (acquisition phase). The escape latency, (i.e., the time required by the rat to find and climb onto the platform), was recorded for up to 120 s. Each rat was allowed to remain on the platform for 30 s, and was then removed to its home cage. If the rat did not find the platform within 120 s, it was manually placed on the platform and returned to its home cage after 30 s. To assess reference memory at the end of learning, a probe trial was given. 24 h after the last acquisition day, the island was removed and the search strategy of the rat was monitored to evaluate whether it used spatial memory to search for the island in the quadrant where it had previously been located. On days 6-7 (days 13-14 from the blast) the platform was placed at the opposite end of the pool, and the rat was retrained in four daily sessions (reversal phase).

### Retrospective Classifications and Re-analyses by Response Patterns

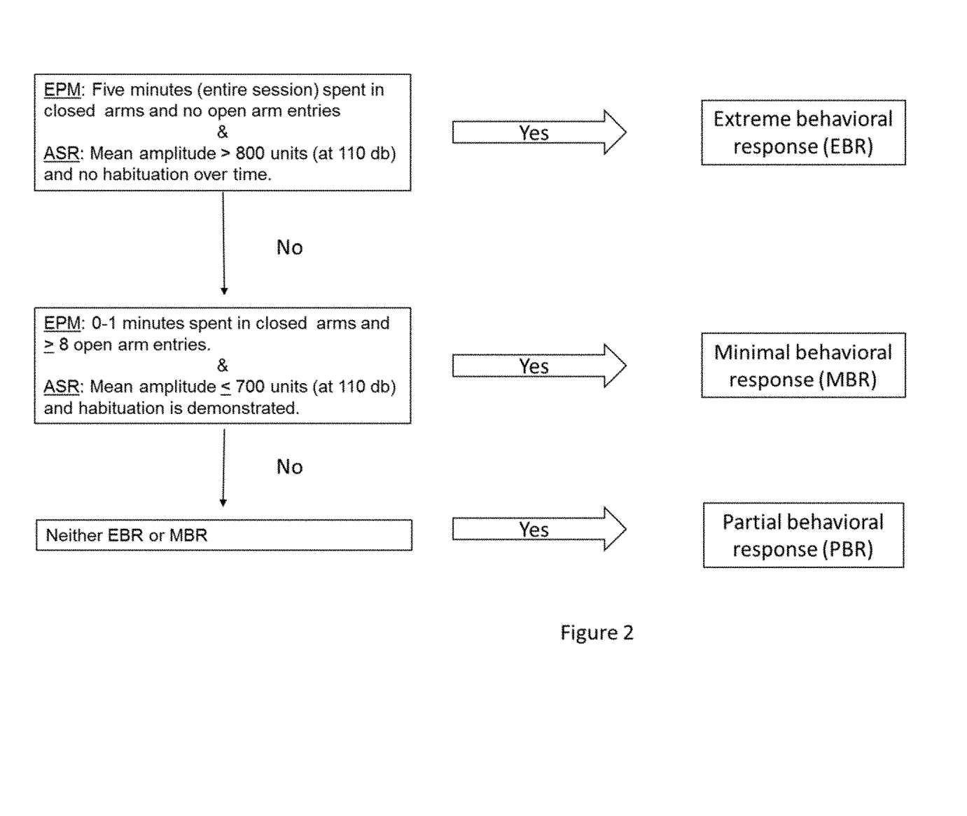

To model DSM (Diagnostic and Statistical Manual of Mental Disorders) criteria for PTSD, the “the cut-off behavioral criteria” (CBC) model of PTSD-like phenotype was employed. This model is based on the understanding that a clinical diagnosis of PTSD is made only if an individual exhibits a certain number of symptoms of sufficient severity from well-defined symptom-clusters over a specific period of time. Classifying the degree of how individual behavior is affected by a stressor is based on the premise that an extreme behavioral response to a priming trigger is inadequate and maladaptive, and represents a pathological response. Animals were classified according to their behavioral response pattern on both the EPM and ASR, by using the CBC, as exhibiting either an “extreme behavioral response” (EBR) or a “minimal behavioral response” (MBR). Behavioral performances that fulfilled neither set of criteria were labeled as exhibiting a “partial behavioral response” (PBR). This procedure is detailed in FIG. 2.

Memory loss or impairment is one criterion for human mTBI. As such, we used the animals' performance in the MWM to evaluate learning and memory. Examination of escape latencies and time spent in different areas of the pool were used to assess learning and memory. Diagnostic criteria that have been previously validated and proven to be reliable were used to determine whether an animal exhibited mTBI behavior.

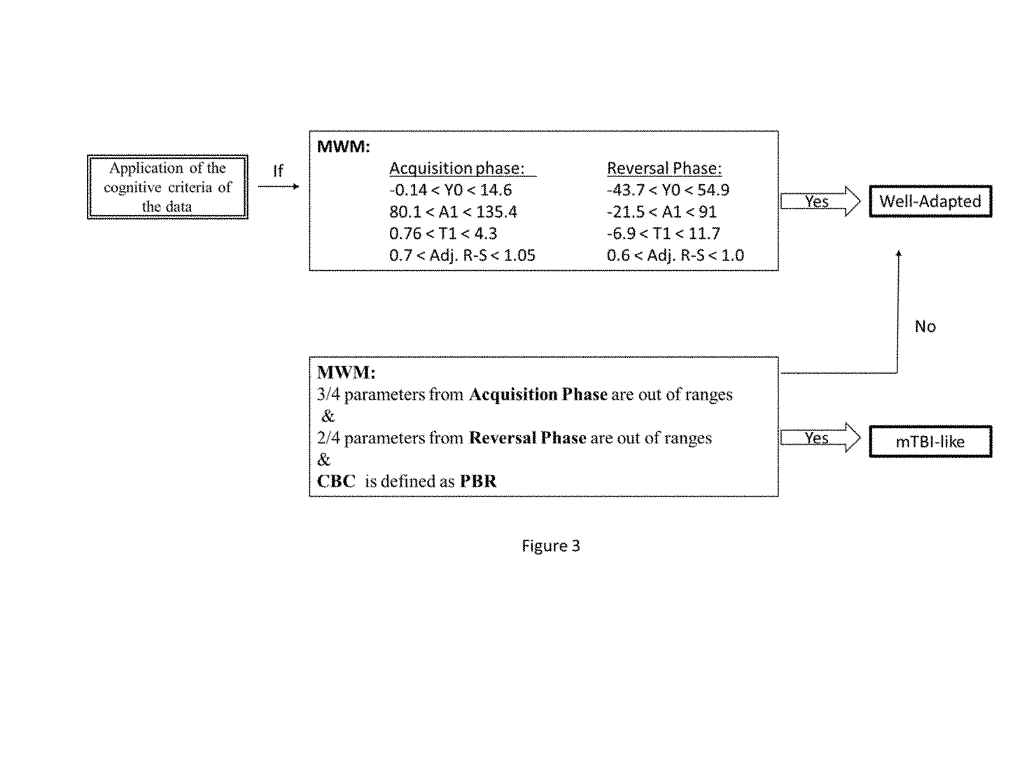

Escape Latency (EL) refers to the time in seconds it took the rat to reach the platform as a function of input number (number of trials); Y₀ refers to the asymptotic time it took the rat to find the platform for large input values (t>>). A refers to the difference (Δ) between the time it took the rat to find the platform on the initial input and the asymptotic time (Y₀). T refers to the decay constant, a measure of the rat's rate of learning. The slope at the initial input (A/t) is the initial learning speed, and the adjusted R² refers to the goodness of fit. Where: EL0=the asymptotic value of EL as t=end point or EL final; ELΔ=the difference between EL peak and EL0; t=number of inputs to the maze. According to the means of the exponential decay parameters (EL0, ELΔ, T and adjusted R-square), confidence limit for “normal performance” (defined for each variable [±2 standard deviations] for acquisition and reversal phases. Subsequently, the memory performance obtained by fitting the escape latency decay curve was used to assess learning and memory performance after blast-exposure. Individual animals were classified according to their cognitive and behavioral response pattern, as exhibiting either “mTBI-related” or well adapted memory performance. The procedure is detailed in FIG. 3.

### Tissue Preparation

All animals were euthanized 24 hr after the last behavioral tests. Animals were deeply anesthetized via an intraperitoneal injection of a ketamine and xylazine mixture (70 mg•kg-1, 6 mg•kg-1, respectively) and perfused transcardially with cold 0.9% physiological saline followed by 4% paraformaldehyde (Sigma-Aldrich) in 0.1 M phosphate buffer (pH 7.4). Brains were quickly removed, postfixed in the same fixative for 12 h at 4° C., and were cryoprotected overnight in 30% sucrose in 0.1 M phosphate buffer at 4° C. Brains were frozen on dry ice and stored at −80° C. Serial coronal sections (10 μm) at the level of dorsal hippocampus were collected for each animal, using a cryostat (Leica CM 1850) and mounted on coated slides.

### Immunofluorescence

Sliced sections were air dried and incubated in frozen methanol (2 min) and in 4% Para-formal-aldehyde (4 min). After three washes in phosphate buffer saline (PBS) containing Tween 20 (PBS/T) (Sigma-Aldrich), the sections were incubated for 60 min in a blocking solution (normal goat serum, in PBS) and then overnight at 4° C. with the primary antibodies against neuropeptide Y (NPY) (mouse monoclonal anti-NPY antiserum (1:500), product code: sc-133080, Santa Cruz Biotechnology, Inc. Heidelberg Germany), brain-derived neurotrophic factor (BDNF) (rabbit polyclonal anti-BDNF antiserum (1:300), product code: sc-ANT-010, Alomone Labs, Jerusalem Israel), glial fibrillary acidic protein (GFAP) (mouse polyclonal anti-GFAP antiserum (1:200), product code: G3893, Sigma-Saint Louis, Mo., USA) and tau protein (rabbit polyclonal anti-Tau antiserum (1:1000), product code: Ab-64193, Abcam Israel). After three washes in PBS/T, sections were incubated for 2 h in DyLight-488 labeled goat-anti-rabbit IgG (BNDF: 1/1250; Tau: 1/1750) or in Dylight-594 goat anti-mouse IgG (NPY: 1/500; GFAP: 1/500; KPL, MD, USA) in PBS containing 2% normal goat or horse serum.

### Quantification

A computer-assisted image analysis system (Leica Application Suite V3.6, Leica, Germany) was used for quantitative analysis of the immunostaining and 50× objective lens were employed to assess the number of BDNF-IR, NPY-IR, GFAP-IR, and Tau-IR protein cells in the hippocampus, divided into three (counted separately) areas: CA1 subfield, CA3 subfield and dentate gyms (DG). The regions of interest were outlined and computer-aided estimation was used to calculate the number of BDNF-IR, NPY-IR, GFAP-IR, and tau-IR protein cells in the pyramidal layer of CA1 and CA3, and in the granular layer of the DG. Seven representative sections of the hippocampus were chosen (between Bregma −2.30 and Bregma −3.60) from each animal, from each group. The sections were analyzed by two observers blinded to the treatment protocol. Standard technique was used to estimate the number of NPY, BDNF, GFAP, and tau protein cell profiles per unit area for each investigated hippocampal structure.

### Measurement of Brain Carnosine and Histidine Content

Carnosine and histidine content in brain homogenates were determined by High Performance Liquid Chromatographic/tandem mass spectrometric (HPLC/MS/MS) analysis according to previously published methods. Brains were partially thawed on ice and five brain regions were sampled: cerebral cortex, hypothalamus, hippocampus, amygdala, and thalamus. Each sample was weighed and transferred into individual vials for further homogenization. Samples were analyzed using HPLC-MS/MS with positive electrospray ionization for histidine and carnosine. Quantification of histidine and carnosine was performed using a deuterium labeled internal standard (IS), L-alanine-d4 and dipeptide alanyl-glutamine. The proteins in the brain tissue samples were removed by the protein precipitation with methanol. After protein precipitation the brain tissue samples were derivatized with 3M hydrogen chloride 1-butanol solution. After addition of internal standards and centrifugation the samples were injected onto the HPLC-MS/MS system. All samples were analyzed separating them first by reversed phase gradient LC and subsequently detecting them using electrospray ionization and multiple reaction monitoring (MRM).

### Statistical Analyses

To compare the effect of β-alanine ingestion on the prevalence of animals exhibiting mTBI-like, PTSD-like and the co-morbid mTBI-PTSD-like characteristics a χ2 analysis was used. Comparisons of brain carnosine and histidine content between BA and PL were performed using an unpaired student's t test. A one-way analysis of variance (ANOVA) was employed to compare immunofluorescence differences of BDNF, NPY, GFAP and Tau protein between BA, PL and CTL. In the event of a significant F ratio, LSD post-hoc analysis was used for pair-wise comparisons. Pearson's product-moment correlation was used to determine selected bivariate correlations. Data were analyzed using SPSS v22 software (SPSS Inc., Chicago, Ill.). All data are reported as mean±SD. An alpha level of p<0.05 was used to determine statistical significance.

### Example I

Prevalence Rates of mTBI, PTSD and mTBI+PTSD According to the Behavioral and Cognitive Performance Criteria

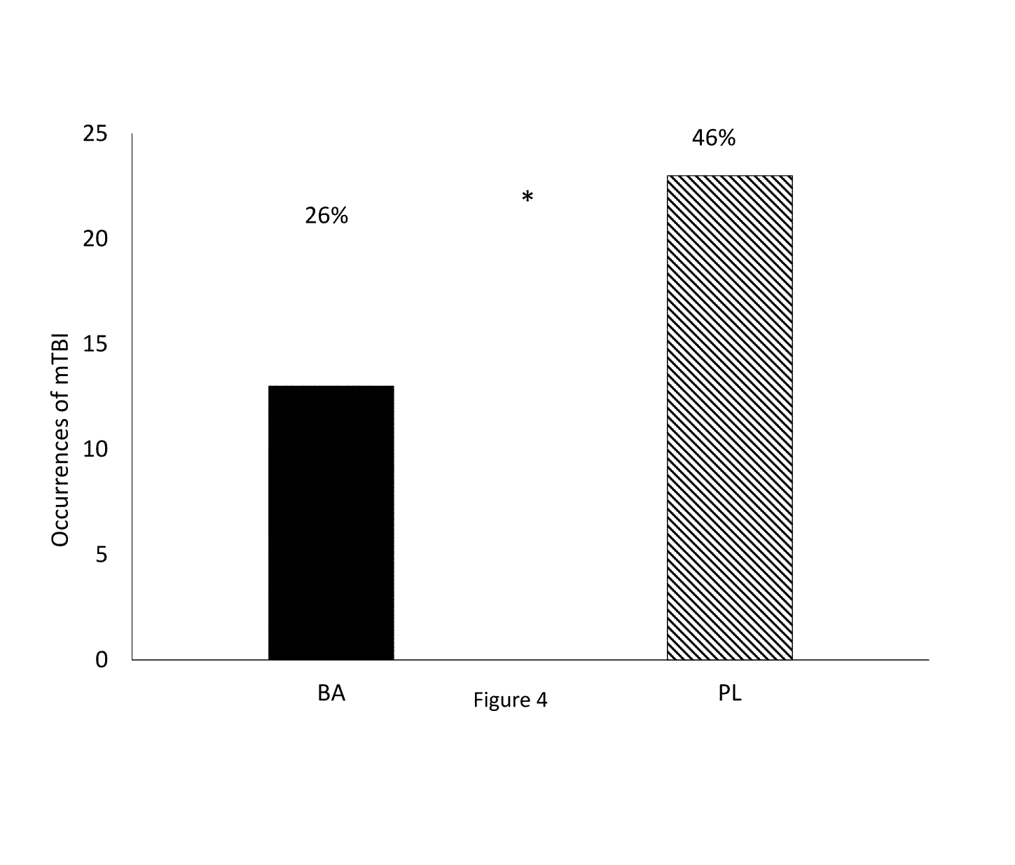

Significant differences were found between groups in the occurrence of animals fulfilling the criteria for mTBI (χ²=4.05, p=0.044) (see FIG. 4). The prevalence of animals demonstrating mTBI was significantly lower in BA than in PL treated rats (26.5% [ 13/49] and 46%, [ 23/50], respectively). No significant differences were noted between the groups in the prevalence of animals displaying PTSD-like patterns of behavior (χ²=0.007, p=0.93). The prevalence of PTSD was similar between BA and PL treated rats (10.2% [ 5/49] and 12.0% [ 6/50], respectively). In addition, the prevalence of animals demonstrating the comorbid pattern of mTBI+PTSD was not significantly different (χ²=1.31, p=0.25) between BA (4%, 2/49) and PL (10%, 5/50). A trend though was noted (χ²=3.07, p=0.08) in the prevalence of well-adapted animals between the groups. Animals in BA exposed to the low pressure blast wave tended to be more well adapted (67%, 33/49) than animals in PL (50%, 25/50).

### Example II

### BDNF Expression at Day 16 Following Blast Exposure

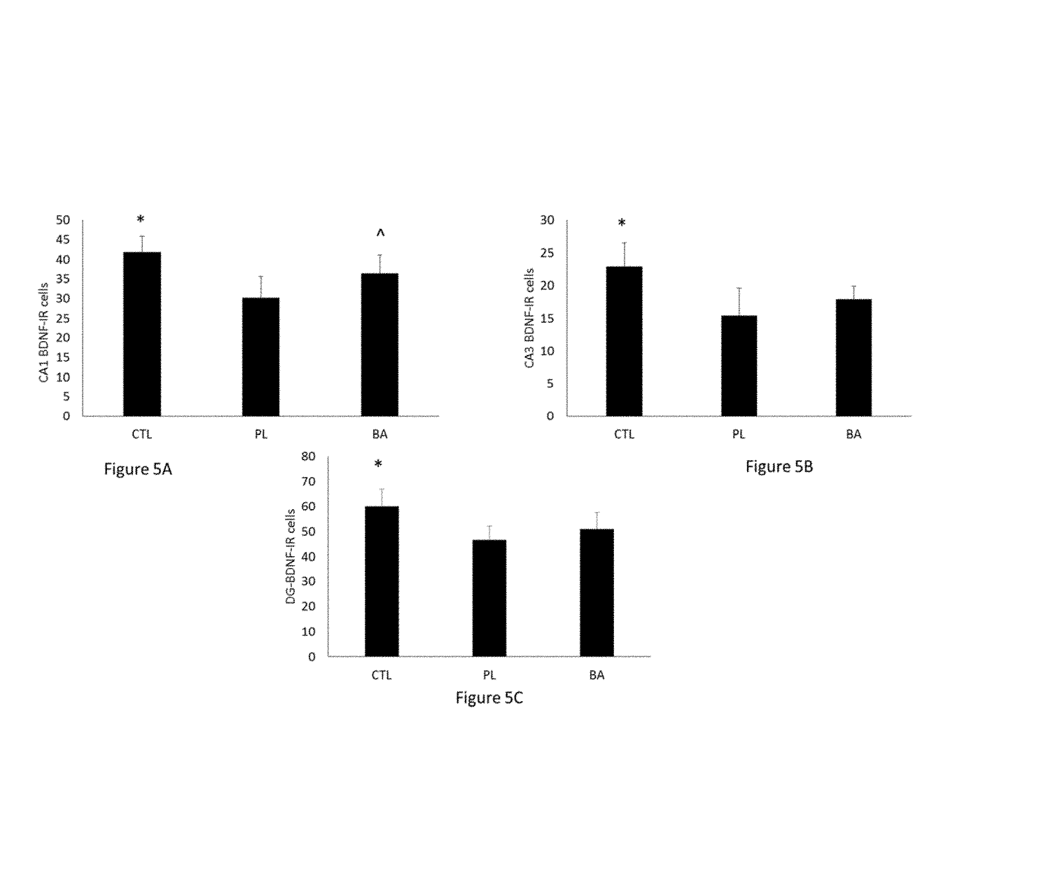

Comparisons between BA, PL and CTL for BDNF expression in the CA1, CA3 and DG subregions can be observed in FIGS. 5A-5C, respectively. Significant differences were noted in the CA1 (F (2, 25)=12.9, p<0.001), CA3 (F (2, 25)=11.1, p<0.001) and DG (F (2, 25)=10.5, p<0.001) subregions. BDNF expression in the CA1 subregion of animals not exposed to the blast and fed a normal diet (CTL) were significantly higher than both PL (p<0.001) and BA (p=0.025). BDNF expression in animals that were exposed to the blast and supplemented with β-alanine were significantly higher than PL (p=0.009). BDNF expression in the CA3 subregion in CTL was significantly greater than both BA (p=0.005) and PL (p<0.001). No difference was noted in BDNF expression in CA3 between BA and PL (p=0.110). In the DG subregion, BDNF expression in CTL was significantly greater than both BA (p=0.005) and PL (p<0.001), while no differences were noted in BDNF expression in the DG subregion between BA and PL (p=0.136).

### NPY Expression at Day 16 Following Blast Exposure

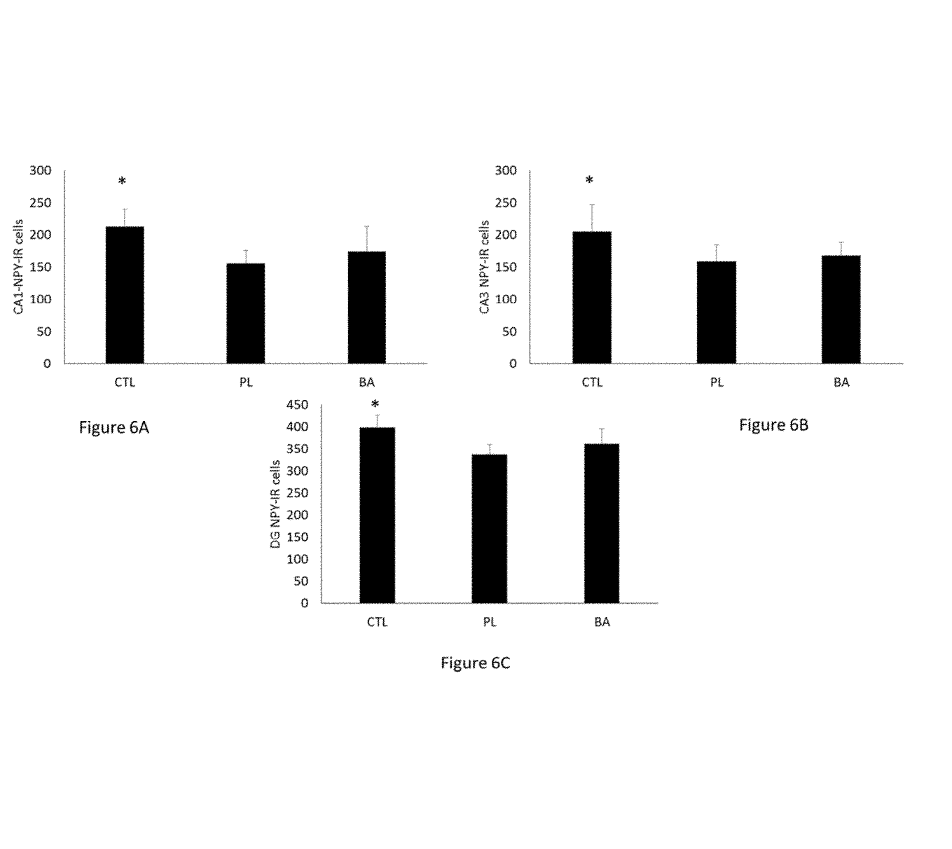

Comparisons between BA, PL and CTL for NPY expression in the CA1, CA3 and DG subregions are depicted in FIGS. 6A-6C, respectively. Significant differences were noted in the CA1 (F (2, 25)=8.3, p=0.002), CA3 (F (2, 25)=5.9, p=0.008) and DG (F (2, 25)=10.2, p=0.001) subregions. NPY expression in the CA1 subregion of CTL were significantly higher than BA (p=0.011) and PL (p<0.001). No significant differences though were noted between BA and PL (p=0.196). NPY expression in the CA3 subregion for CTL was significantly greater than both BA (p=0.032) and PL (p=0.003), and again no differences were noted between BA and PL (p=0.480). In the DG subregion, NPY expression in CTL was significantly greater than both BA (p=0.011) and PL (p<0.001), while NPY expression in BA trended (p=0.074) towards a higher response compared to PL.

### GFAP Expression at Day 16 Following Blast Exposure

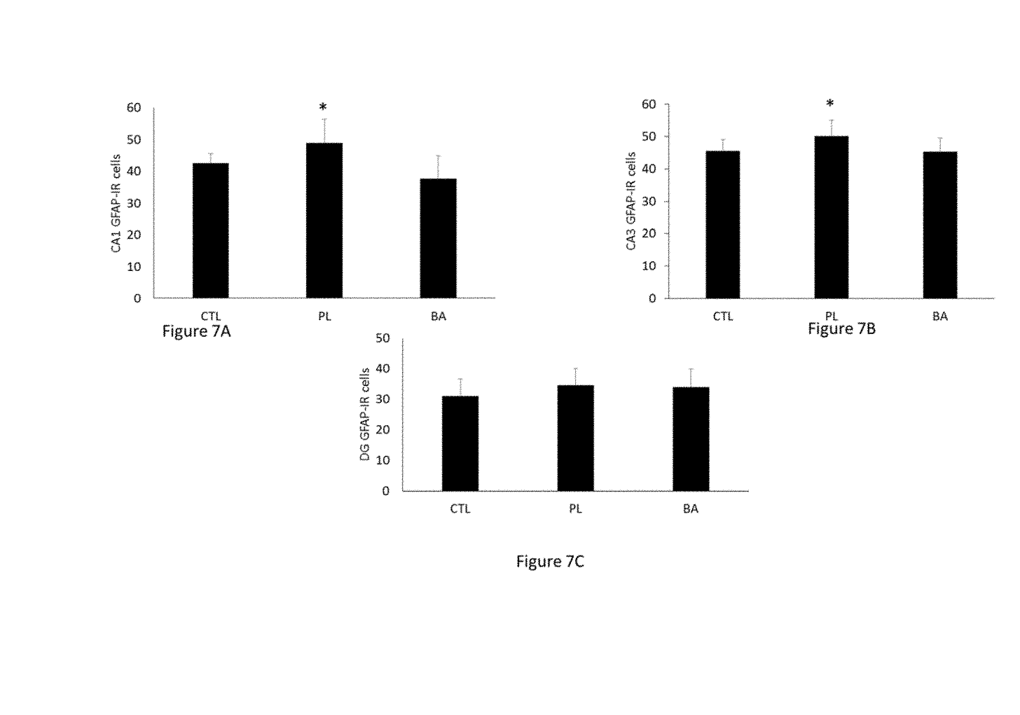

Comparisons between BA, PL and CTL for GFAP expression in the CA1, CA3 and DG subregions are depicted in FIGS. 7A-7C, respectively. Significant differences were noted in the CA1 (F (2, 25)=7.6, p=0.003) and CA3 (F (2, 25)=3.7, p=0.040) subregions, but no difference was noted in the DG (F (2, 25)=0.99, p=0.387) subregion. GFAP expression in the CA1 subregion of PL was significantly greater than BA (p=0.001) and CTL (p<0.047). No differences were noted between CTL and BA (p=0.126). GFAP expression in the CA3 subregion for PL was significantly greater than both BA (p=0.021) and CTL (p=0.040), but no differences were noted between BA and CTL (p=0.876).

### Tau Protein Expression at Day 16 Following Blast Exposure

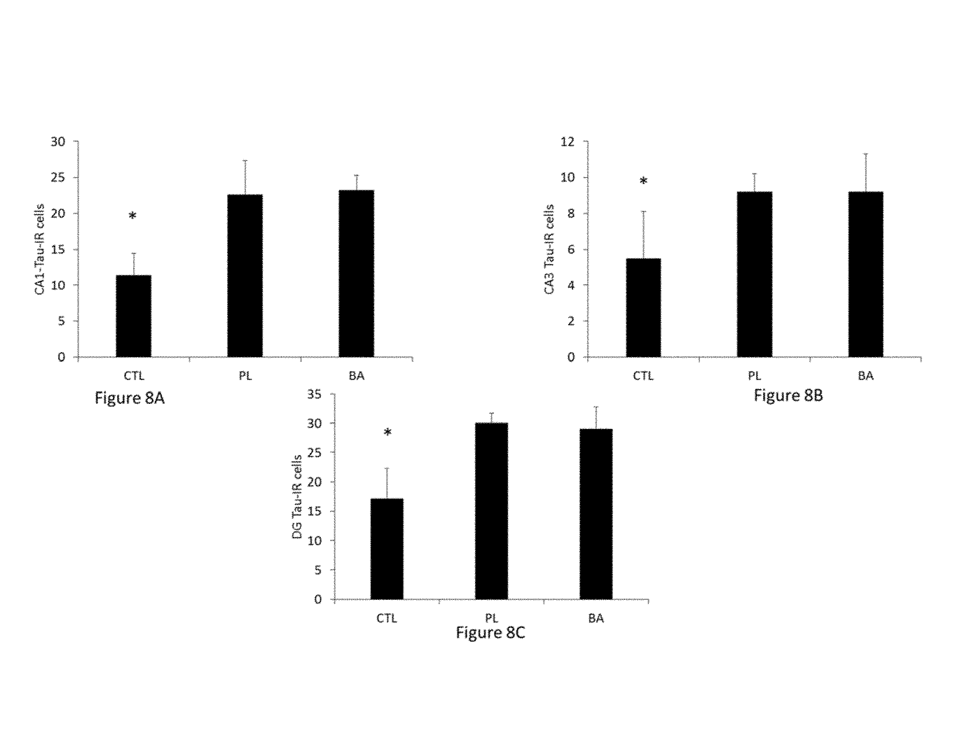

Comparisons between BA, PL and CTL for tau protein expression in the CA1, CA3 and DG subregions are depicted in FIGS. 8A-8C, respectively. Significant differences were noted in the CA1 (F (2, 27)=37.2, p<0.001), CA3 (F (2, 27)=11.0, p<0.001) and DG (F (2, 27)=36.5, p<0.001) subregions. Tau protein expression in the CA1 subregion of CTL were significantly lower than both BA (p<0.001) and PL (p<0.001). No significant differences though were noted between BA and PL (p=0.700). Tau protein expression in the CA3 subregion for CTL was also significantly lower than both BA (p<0.001) and PL (p<0.001), and again no differences were noted between BA and PL (p=1.000). In the DG subregion, tau protein expression in CTL was significantly lower than both BA (p<0.001) and PL (p<0.001), with no differences observed (p=0.557) between BA and PL.

### Example III

Brain Carnosine and Histidine Content at Day 16 Following Blast exposure

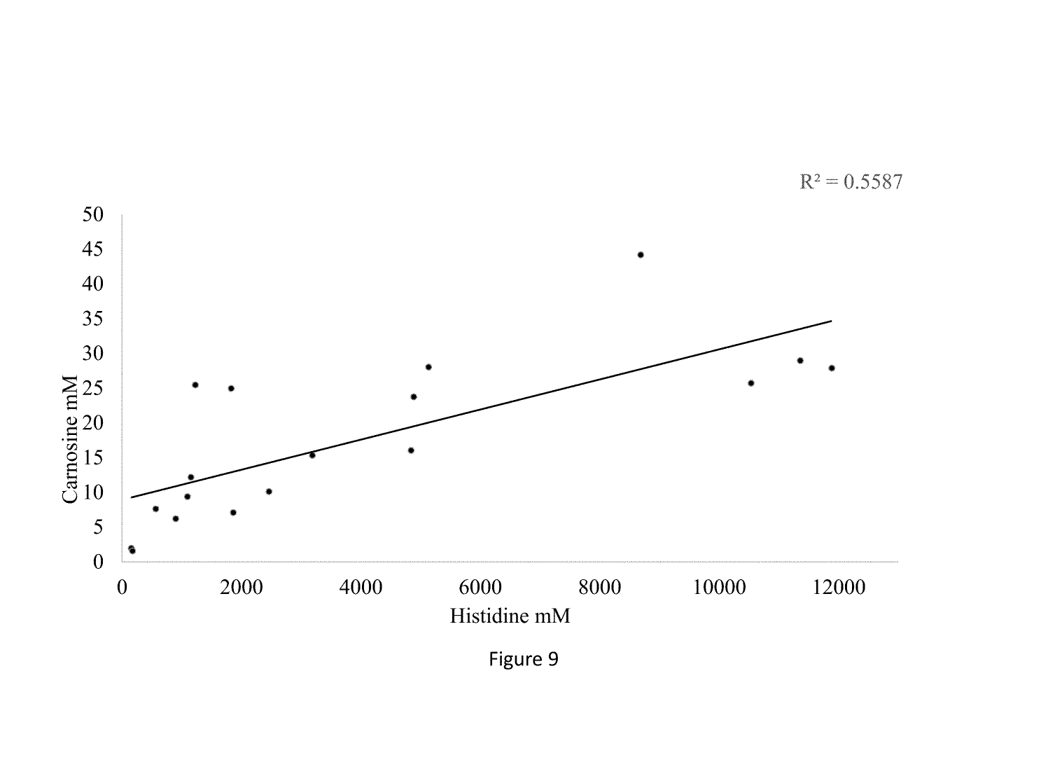

Differences in carnosine and histidine content in various areas of the brain can be observed in Table 1. Across the five brain regions analyzed the carnosine content in animals that consumed β-alanine was on average 79% higher than in those animals that consumed the vehicle. Carnosine content in the cerebral cortex was significantly higher (p=0.048) for BA compared to PL. Trends towards a difference were also seen in the hippocampus (p=0.058) and amygdala (p=0.061). Histidine content across the five regions of the brain that were analyzed was 86% higher for BA than P. Trends towards a higher histidine content were noted in the hippocampus (p=0.053), cerebral cortex (p=0.070) and thalamus (p=0.108) for BA compared to PL. Carnosine content was significantly correlated (r=0.75, p<0.001) to histidine content in the hippocampus (see FIG. 9). Positive correlations were also noted between carnosine and histidine in the cortex (r=0.48, p=0.037), hypothalamus (r=0.48, p=0.036) and thalamus (r=0.43, p=0.070).

TABLE 1
Brain Carnosine and Histidine Concentrations (mM)

| | | Hippocampus | Cortex | Hypothalamus | Amygdala | Thalamus |
|---|---|---|---|---|---|---|
| Carnosine | BA | 22.4 ± 11.3 | 364 ± 178 | 22.7 ± 26.2 | 23.2 ± 18.4 | 50.9 ± 33.9 |
| | PL | 12.7 ± 8.4 | 213 ± 110 | 9.5 ± 6.7 | 10.4 ± 4.4 | 57.5 ± 63.1 |
| | p value | 0.058 | 0.048* | 0.166 | 0.061 | 0.795 |
| Histidine | BA | 5632 ± 4995 | 5669 ± 4471 | 1862 ± 1794 | 2619 ± 1467 | 26025 ± 21754 |
| | PL | 2768 ± 1771* | 2128 ± 1537 | 1144 ± 794 | 3029 ± 1582 | 12141 ± 12523 |
| | p value | 0.053 | 0.070 | 0.266 | 0.576 | 0.108 |

All data reported as mean ± SD;
*Significant difference between groups.

What is claimed is:

1. A method of reducing the prevalence of mild traumatic brain injury (mTBI) in an individual comprising administering a dietary supplement containing a free amino acid beta-alanine to an individual at risk of experiencing mTBI thereby reducing the prevalence of mTBI in said individual.

2. The method of claim 1, wherein the mTBI is caused by a blast wave or by a blow to individual's head.

3. The method of claim 1, wherein the dietary supplement further comprises at least one of a carbohydrate, a creating, a nitrate, a glycine, or a free amino acid histidine.

4. The method of claim 1, wherein the free amino acid beta-alanine is in a sustained release formulation.

5. A method of maintaining brain function in an individual at risk of mTBI comprising administering a dietary supplement containing a free amino acid beta-alanine to an individual at risk of experiencing mTBI maintaining brain function in said individual.

6. The method of claim 5, wherein the mTBI is caused by a blast wave or a blow to said individual's head, wherein said individual is selected from the group consisting of soldiers, first responders, construction workers, and athletes.

7. The method of claim 5, wherein the dietary supplement further comprises at least one of a carbohydrate, a creating, a nitrate, a glycine, or a free amino acid histidine.

8. The method of claim 5, wherein the free amino acid beta-alanine is in a sustained release formulation.

9. A method of maintaining BDNF expression in the hippocampus of an individual at risk of mTBI comprising administering a dietary supplement containing a free amino acid beta-alanine to an individual at risk of experiencing mTBI thereby maintaining BDNF expression in the hippocampus of said individual.

10. The method of claim 9, wherein the mTBI is caused by a blast wave or a blow to said individual's head, wherein said individual is selected from the group consisting of soldiers, first responders, construction workers, and athletes.

11. The method of claim 9, wherein the dietary supplement further comprises at least one of a carbohydrate, a creating, a nitrate, a glycine, or a free amino acid histidine.

12. The method of claim 9, wherein the free amino acid beta-alanine is in a sustained release formulation.

13. A method of maintaining GFAP expression in the hippocampus of an individual at risk of mTBI comprising administering a dietary supplement containing a free amino acid beta-alanine to an individual at risk of experiencing mTBI thereby maintaining GFAP expression in the hippocampus of said individual.

14. The method of claim 13, wherein the mTBI is caused by a blast wave or a blow to said individual's head, wherein said individual is selected from the group consisting of soldiers, first responders, construction workers, and athletes.

15. The method of claim 13, wherein the dietary supplement further comprises at least one of a carbohydrate, a creating, a nitrate, a glycine, or a free amino acid histidine.

16. The method of claim 13, wherein the free amino acid beta-alanine is in a sustained release formulation.

17. A method of maintaining memory function in an individual at risk of mTBI comprising administering a dietary supplement containing a free amino acid beta-alanine to an individual at risk of experiencing mTBI thereby maintaining memory function of said individual.

18. The method of claim 17, wherein the mTBI is caused by a blast wave or a blow to said individual's head, wherein said individual is selected from the group consisting of soldiers, first responders, construction workers, and athletes.

19. The method of claim 17, wherein the dietary supplement further comprises at least one of a carbohydrate, a creating, a nitrate, a glycine, or a free amino acid histidine.

20. The method of claim 17, wherein the free amino acid beta-alanine is in a sustained release formulation.
